(19) United States
(12) Patent Application Publication
(10) Pub. No.: US 2022/0338813 A1
(43) Pub. Date: Oct. 27, 2022

(54) IMPLANTABLE ELECTRONIC SENSING SYSTEM FOR MEASURING AND MONITORING MEDICAL PARAMETERS

(71) Applicant: UNIVERSITAT POMPEU FABRA, Barcelona (ES)

(72) Inventors: Antoni IVORRA CANO, Barcelona (ES); Quim CASTELLVÍ FERNÁNDEZ, Barcelona (ES); Laura BECERRA FAJARDO, Barcelona (ES)

(21) Appl. No.: 17/640,568

(22) PCT Filed: Jul. 13, 2020
(86) PCT No.: PCT/EP2020/069688

§ 371 Date: Mar. 4, 2022

(30) Foreign Application Priority Data
Sep. 4, 2019 (EP) 19382757.3

(51) Int. Cl. A61B 5/00; A61B 5/021; A61B 5/0215; A61B 5/277

(52) U.S. Cl. CPC A61B 5/6861; A61B 5/02141; A61B 5/0215; A61B 5/277; A61B 5/0031; A61B 2562/0247; A61B 2562/162; A61B 2562/0214

(57) ABSTRACT

The invention refers to an implantable sensing system comprising an electronic implant and a reading unit to obtain measurements originating at the implant or its surroundings to characterize physical and/or chemical clinical parameters of a living body. The electronic implant comprises an electronic circuit and at least two electrodes connected to the electronic circuit. The electronic circuit comprises a capacitor and a device of asymmetric conductance capable of rectifying an alternating current, both connected in series between two electrodes. An electronic component is connected in parallel with the device of asymmetric conductance, for the capacitor discharge. The capacitor, the device of asymmetric conductance and/or the electronic component, can be a transducer selected such as an operational parameter of the transducer is variable depending on a physical and/or chemical condition of a medium of a living body. The implant features a minimal invasiveness, such as it can be implanted by injection or by catheterization rather than by open surgery.

### OBJECT OF THE INVENTION

The present invention generally refers to implantable sensing systems for measuring and monitoring medical parameters from a living human or animal body.

More specifically, the invention refers to an electronic implant and to a reading unit to obtain measurements originating at the implant or its surroundings to characterize physical and/or chemical clinical parameters of a living body for medical and biomedical applications.

An object of the invention is to provide an electronic implant of reduced dimensions, that features a minimal invasiveness during its implantation, in such a way that the implant can be implanted by injection or by catheterization rather than by open surgery.

The system of the invention can advantageously be used for example for monitoring: congestive heart failure, blood pressure in vessels, pulse oximetry, blood glucose, tissue ischemia, and tissue edema among others.

### BACKGROUND OF THE INVENTION

Implantable sensing systems have been developed to measure and monitor clinically relevant magnitudes in a living body for medical applications such as: blood glucose for diabetic patients, pH for monitoring gastroesophageal reflux, blood pressure for monitoring heart failure, bladder pressure in urinary incontinence patients etc. In contrast to external sensing systems, implantable systems are capable of detecting the stimuli where they originate and that provides them with higher accuracy.

These implantable sensing systems are normally used for diagnosis and for determining treatment dosage and timing. Surprisingly, the number of commercially available products is very reduced compared with the large amount of published academic research in the field of implantable sensors. Such disparity is due to market and regulatory constraints and, more importantly, to practical factors such as the invasiveness of the available implants.

Therefore, implantable sensing systems that are minimally invasive either because the implantation of the devices is done by catherization or by injection, or because the systems re-use an already surgically implanted device, are by far, preferred over systems that require highly invasive surgeries, even if the performance of the non-invasive products is lower than the surgically implanted ones.

A known type of electronic sensing implants are based on active electronics. These implants incorporate a mechanism to generate electric energy to power an electronic circuit capable of reading and processing signals from a sensor, and transmitting the result to an external unit for further processing or representation. In some cases, the electric power is entirely generated internally (e.g. with electrochemical batteries) and in other cases the electric power is either generated by transforming a sort of energy already present in the body (e.g. so-called energy harvesters that can transform kinematic energy into electric energy), or by wireless power transmission from an external unit (e.g. by ultrasound power transmission or by inductive coupling power transmission).

In the above cases, the mechanism for generating the electric energy requires bulky components that hinder miniaturization of the implants, in such a way that implantation by catheterization or injection is not feasible.

Another type of known electronic sensing implants are those based on passive electronics, that are those that do not contain a mechanism to power a circuit. Most of the commercially available systems of this type are based on combinations of inductors and capacitors (LC systems) that resonate at a specific frequency when an alternating magnetic field is applied. Such frequency is typically determined by a capacitor which acts as the sensor, as its capacitance depends on the magnitude of interest. The main disadvantage of these systems is that they require coils with a relatively large diameter, both at the implant and at the external unit, especially if the device is intended for deep implantation.

In non-electronic sensing implants, the magnitude of interest is transduced into a non-electrical magnitude which is read and processed by a reading unit. Some available systems of this type are based on transduction into optic properties and, in particular, into fluorescence. These systems exhibit some significant drawbacks, for instance, since these systems use chemical reactions, the operating life of the implants is short. In addition, these systems are limited to applications in which the sensor is very close to the reading unit, so that optical transmission is feasible.

Implantable sensing systems may be of use for blood pressure monitoring. Long term and continuous blood pressure monitoring is required for a number of clinical needs, which are unmet by conventional blood pressure measurement systems based on sphygmomanometers or catheter pressure sensors. These two technologies are too obtrusive for long term and continuous blood pressure monitoring. Attempts have been carried out to implement implantable pressure sensors to monitor blood pressure in a non-obtrusive way. However, until now the developed implantable systems are relatively bulky and are not adequate for deployment in the narrow arteries and veins of the peripheral vascular system which would be preferable over implantation in vessels of the abdomen or the thorax for minimizing risks.

Another case in which implantable sensing systems may be of use is for detecting congestive heart failure based on impedance measurements of the lungs. Existing implantable pacemakers and defibrillators incorporate a similar functionality: they provide measurements of the so-called transthoracic impedance for early detection of worsening heart failure. Those measurements are obtained by performing impedance measurements across the electrodes they possess for their therapeutic function. However, since these electrodes are implanted within large blood cavities (i.e. the heart ventricles and auriculas) and the conductivity of the blood is much higher than that of lung tissues, the measurements that these systems perform exhibit low sensitivity to the conductivity of the lungs and, consequently, they exhibit a large rate of false positives. Furthermore, it must be noticed that only a minority of patients with congestive heart failure are implanted with a defibrillator or a pacemaker.

In the scientific publications (Conf. Proc. ICNR2014 447-455 doi: 10.1007/978-3-319-08072-7_67, J Neural Eng. 2015 12(6):066010 doi: 10.1088/1741-2560/12/6/066010) and the U.S. Pat. No. 9,446,255 the use of implants for stimulation is disclosed. However, these publications only relate to stimulation, they are silent to the use of these implants for measurement or monitoring parameters in a living body.

U.S. Pat. Nos. 8,725,270 and 8,909,343 describe the use of implants based on a single diode for sensing the impedance of tissues and biopotentials, by processing the harmonics generated by the diode when a radiofrequency electromagnetic wave is applied. In these patents, since the diode is the only sensing element, there are constrains with the magnitudes that can be sensed, and the accuracy of the measurements is reduced.

It is known to use the volume conduction property of human tissue as a natural medium for delivery of energy. For example, the PCT publication WO2006105245 (A2) discloses the use of the volume conduction for energy delivery to implants. The disclosure of this PCT publication is focused on an external antenna design, that consists of an array of electrodes arranged to receive voltage and work collaboratively to transmit electrical energy to a target site, wherein the external delivery of electrical energy from outside the human body to a target site within the human body is carried out, such as electrical stimulation of muscles and power delivery to implanted devices.

This PCT publication is silent to the use of electronic rectification of volume conducted currents to obtain measurements originating at the implant or its surroundings to characterize physical and/or chemical clinical parameters of a living body for medical and biomedical applications. It is also silent about the structure of the implants.

Therefore, there is the need in this technical field to further reduce the dimensions and invasiveness of implantable sensing systems, in such a way that these systems can be easily deployed by injection or by catheterization.

### SUMMARY OF THE INVENTION

The invention is defined in the attached independent claim 1, including the preferred embodiments defined in the dependent claims. The invention satisfactorily solves the drawbacks of the prior art, by providing an implant that exhibits reduced invasiveness, with a thickness ranging from few millimeters to fractions of a millimeter (e.g. 0.5 mm), such as the implant can be deployed by injection or by catheterization.

The invention refers to a sensing system comprising at least one implant and a reading unit cooperating with the implant to obtain accurate measurements to characterize physical and/or chemical clinical parameters of a living body.

An aspect of the invention refers to an implant comprising an electronic circuit and at least two electrodes connected to the electronic circuit, wherein the circuit comprises a capacitor and a device of asymmetric conductance, both connected in series between two electrodes. The device of asymmetric conductance is a two-terminal electronic component or system capable of controlling the direction of current flow through its two terminals, for example a diode, a p-n junction of a transistor or a smart diode.

The capacitor prevents that dc currents flow through the implant, as these dc currents would cause irreversible electrochemical reactions at the implant electrodes, that in turn would damage both the electrodes and the living tissues.

The implant circuit additionally comprises a discharge network connected in parallel with the device of asymmetric conductance for the capacitor discharge, wherein the discharge network comprises at least one electrical or electronic component, for example a resistor and/or a semiconductor device, so that the capacitor discharges through the discharge network and through a medium of a living body in which the implant is immersed.

According to the invention, the capacitor, the device of asymmetric conductance and/or the electrical or electronic component of the discharge network, can be a transducer selected so that one of its operational parameters is variable depending on a physical and/or chemical condition of a medium in which the implant might be implanted for measuring a medical parameter. The medium consists of a tissue or a fluid (e.g. blood) of a human or animal body.

For example, the capacitor or the resistor of the discharge network, can be implemented as a transducer whose characteristic value (capacitance (C) and resistance (R), respectively) depends on a physical or chemical magnitude of interest (i.e. measurand), and a reading unit can derive the value of those magnitudes (i.e. measurements) by processing the characterization. It is well known that if the function that relates the measurand (x) and the transducer output is established (y=f(x)), it is possible to compute a measurement by applying the inverse function (x′=f⁻¹(y)).

The separation distance between the implant electrodes should be large enough so as to pick up a voltage difference sufficient for the operation of the implant circuit. That is, the separation distance must be larger than the minimum voltage for operation, divided by the expected electric field magnitude at the location of the implant.

Preferably, the device of asymmetric conductance is a diode that requires a minimum voltage in the order of hundreds of millivolts for operation. Taking into account that the maximum allowable electric field amplitude at the location of the implant will be in the order of a few volts per centimeter, the diode voltage requirement translates in a minimum inter electrode distance in the order of a few millimeters. Considering that, when implanted, the implant might not be perfectly aligned with the electric field and other uncertainties, the separation distance between the electrodes, and hence the length of the implant, will be in the order of a centimeter or a very few centimeters, preferably the length of the implant is within the range 0.5-5 cm.

In a preferred embodiment, the discharge network consists in a resistor of a given nominal value, and the capacitor is a transducer whose capacitance is variable depending on a physical and/or chemical condition of the medium. Preferably, the capacitor is a pressure capacitor whose capacitance depends on the pressure applied to a part of the capacitor.

In another preferred embodiment, the capacitor has a given nominal capacitance, and the electrical or electronic component of the discharge network, is a resistive transducer, preferably a thermistor or a light dependent resistor (LDR).

In another preferred embodiment, the discharge network contains a current controlling device, such as a current-limiting diode or a JFET current limiter, that controls the discharge current and makes it independent of the impedance of the medium. Such current controlling device may be of nominal current or may be configured to depend on a measurand. For instance, the resistor in a JFET current limiter may be a resistive transducer.

In another preferred embodiment, the implant is adapted for sensing biopotentials or chemical species. In this case, the capacitor has a given nominal capacitance and the electronic component of the discharge network is a transistor configured as a transducer in parallel with the device of asymmetric conductance. The gate or base terminal of the transistor, is in contact with the medium in such a way that the conductance of the transistor depends on voltage gradients at the medium or on the concentration of an ion in the medium. For sensing ion concentrations, the gate or base terminal of the transistor is in contact with the medium directly or through a so-called ion selective membrane. For sensing voltage gradients, the gate or base terminal of the transistor is in contact with the medium through a third electrode.

In this embodiment, the discharge network may additionally comprise an auxiliary diode connected in series with the transistor. The anode of the auxiliary diode is connected to the cathode of the device of asymmetric conductance and to one terminal of the capacitor, and the cathode of the auxiliary diode is connected to the collector or drain of the transistor. The source or emitter of the transistor is connected to the anode of the device of asymmetric conductance, and to one electrode of the implant.

In another preferred embodiment, the device of asymmetric conductance and the electrical or electronic component of the discharge network, cooperate to configure an optical transducer together with an optical reactive material, in such a way that an optical property of the optical transducer is variable depending on a physical or chemical condition of the medium. The optical reactive material, preferably a fluorescence or a phosphorescence variable material, is arranged in the implant to be in contact with the medium when the implant is in use.

In this embodiment, the device of asymmetric conductance is a light emitting semiconductor device that emits light when a current passes through it, and the electronic component of the discharging network is a light sensitive (receiving) conductive device, that allows current flow when it receives a suitable intensity of light. The optical reactive material is arranged to transmit light from the light emitting semiconductor device or to generate light after being illuminated by the light emitting semiconductor device, to the light sensitive conductive device, so that the capacitor can discharge through the light sensitive conductive device, when this device is activated by the light received from the optical reactive material.

The light emitting semiconductor device and the light sensitive (receiving) conductive device, can be implemented as optoelectronic components, for example as a photoemitter and a photodetector (photodiode) respectively. In order to increase the selectivity of this type of implants based on optoelectronic components and, in particular, when the fluorescence of the transducer material is characterized, preferably optical filters or diffraction grids are placed on the photoemitter and on the photodetector (photodiode) in order to select specific light wavelengths or bands of operation.

In another preferred embodiment, the device of asymmetric conductance is a diode, the capacitor has a fixed nominal value, and the implant further comprises a transmitting, reflecting, or refractive optical reactive material. The discharge network comprises a light emitting semiconductor device and a light sensitive conductive device, both connected in parallel with the device of asymmetric conductance. The optical material is arranged to transmit light from the light emitting semiconductor device to the light sensitive conductive device, so that the capacitor can be discharged through the light sensitive conductive device to emit light during the capacitor discharge.

Preferably, for the above optical embodiments, the implant includes a capsule where the electronic implant circuit is housed, and at least a part of the capsule is formed with the optical reactive material described above.

In another preferred embodiment, the optical reactive material is a deformable material whose optical transmissivity varies depending on the pressure or force applied to it, thus, the implant circuit operation depends on the pressure or force applied externally to the capsule.

In another preferred embodiment, the implant is suitable to be deployed inside an artery or a vein for measuring blood pressure. In this case, the implant comprises a capsule having at least a part made of a flexible material that allows pressure transmission from the exterior to the capsule interior. The implant circuit is hermetically housed within the capsule, and the two electrodes of the implant pass through the capsule and are connected to the implant circuit. Preferably, each electrode is a flexible wire conformed as a loop.

In another preferred embodiment, the capacitor and the flexible part of the capsule, are arranged relative to each other such as the capacitance varies with the deformation of the flexible material.

Another aspect of the invention refers to an implantable sensing system comprising at least one electronic implant as any one of the alternative implant configurations described above, and a reading unit for interrogating the electronic implant or implants deployed in a living body. The reading unit comprises: two or more electrodes like surface or skin electrodes, an alternating voltage generator to generate an alternating voltage across the electrodes, and a control and processing module for controlling the voltage generator. The reading unit is configured for emitting bursts of an alternating current, preferably high frequency currents, suitable to reach an implant deployed internally inside a body by volume conduction (galvanic conduction) through the body.

The reading unit is additionally adapted to interrogate an implant by delivering bursts of high frequency electric currents through electrodes. The measurement is obtained by processing the voltage or current signals that result during or after the delivery of the bursts. The voltage signals are measured from the same two electrodes that deliver the current of electric currents, or alternatively from the electrodes in contact with the tissues where the implant is deployed.

Preferably, the implant is a thread-shaped and flexible body with two electrodes at opposite ends, to allow minimally invasive percutaneous deployment.

The invention provides alternative implant circuit architectures, adapted for capturing different measurements. These circuit architectures are implemented either by using discrete components mounted on a micro-circuit board, or by integrating them monolithically in an integrated circuit or microsystem using semiconductor manufacturing technology.

The reading or interrogation method between the reading unit and an implant, comprises the following stages:

1.—applying bursts of alternating current, for example sinusoidal, generated by the reading unit that reach an implant by volume conduction,

2.—detecting resulting voltage and/or current signals at the reading unit,

3.—processing those signals to characterize the capacitance of the implant capacitor, the resistance of the discharging element, or the impedance of the tissues that surround the implant and

4.—processing the characterization for obtaining a measurement of interest.

Preferably, the reading unit is a battery powered hand-help unit, embodied as an external device. Alternatively, the reading unit is formed by two parts, namely: an implantable part (e.g. battery or inductively powered) for reading an implant inside a body, and an external part wirelessly communicated with the implantable part. In particular, the implantable part can consist of a subcutaneously implanted sub-unit capable of generating current bursts and also for taking measurements. In turn, the external sub-unit is capable of processing data wirelessly transmitted by the implanted sub-unit, and for representing the measurements for example in a display and for generating alarms.

Multiple implants can be independently interrogated if they are sufficiently separated within the body. The separation must be enough to ensure that only the implant of interest is powered or activated when the bursts are delivered. That is, in the case that the implants are not intended to be interrogated, it should be ensured that the electric field that reaches them is low enough to prevent conduction of the device of asymmetric conductance (e.g. diode).

Alternatively, multiple implants can be simultaneously energized for operation and selectivity can be achieved by the relative location of the voltage pick-up electrodes of the reading unit.

For all the embodiments of the invention, the injected alternating currents for interrogation, which are either current controlled or voltage controlled, are specially selected to be innocuous to the body. This is accomplished by ensuring that these currents are of sufficient frequency to prevent unsought stimulation of excitable tissues (first requirement), and their power is low enough to prevent excessive heating of tissues due to Joule heating (second requirement).

The first requirement can be readily met by using currents whose power spectral density is well above 100 kHz. For instance, sinusoidal currents with a frequency (f) of or above 1 MHz, are desired. Furthermore, frequencies below 100 MHz are preferred, to prevent that the skin effect becomes significant, and the operation of implants at deep locations is hindered.

The second requirement is achieved by delivering short bursts. For a given voltage gradient (E) required to ensure operation of the implants (e.g. 200 V/m), burst duration (B) and burst repetition frequency (F), are selected to ensure that power dissipation in the tissue where the implants are located does not reach a safety threshold.

Safety thresholds for electromagnetic power dissipation in tissues are usually specified by the so-called Specific Absorption Rate (SAR), which is measured in W/kg. SAR can be calculated with the following expression:

$$SAR = \frac{{σ ( {E}_{RMS} )}^{2}}{ρ}$$

where σ is the electrical conductivity of the tissue (S/m), ρ is the mass density of the tissue (kg/m³) and E_{RMS} is the root mean square value of the electric field in the tissue (V/m). From that expression it can be obtained the following requirement for F and B in the case of sinusoidal bursts:

$$FB < \frac{2 {ρ SAR}_{MAX}}{{σ E}^{2}}$$

A SAR value of 2 W/kg is considered to be a safe threshold in all circumstances according to different standards. If the above expression is particularized for the case of muscle tissue at 10 MHz (σ_{10 MHz}=0.62 S/m, ρ=1060 kg/m³) and it is assumed that the field required for operation is 200 V/m, the 2 W/kg limit yields FB<0.17 s/s. Thus, for instance, if the burst duration, is 10 μs, the maximum repetition frequency would be 17 kHz.

It must be noted that the SAR limitation must not only be met where the implants are located but also in all tissue regions where the interrogation currents flow through. Therefore, since current densities (and voltage gradients) will be probably higher in the vicinity of the current injecting electrodes of the reading unit, the FB product will have to be scaled down.

Preferably, the interrogation signal consists of a sinusoidal waveform with a frequency between 100 kHz and 100 MHz which is delivered as bursts with a duration between 0.1 ρs and 10 ms, and a repetition frequency between 0 Hz (i.e. single burst interrogation) and 100 kHz.

Some of the advantages of the system of the invention are summarized below:
minimal invasiveness during its implantation, as the implant can be deployed by injection or by catheterization,
implantation within body locations that were not feasible before,
accurate measurements compared with external measurement devices,
low cost implants and simplification of external reading unit.

### BRIEF DESCRIPTION OF THE DRAWINGS

Preferred embodiments of the invention, are henceforth described with reference to the accompanying drawings, wherein:

FIG. 1.—shows a schematic representation of the implantable sensing system of the invention, wherein an implant is shown deployed inside a body while it is being interrogated by an external reading unit.

FIG. 2.—shows an electric diagram of a preferred circuit architecture for an implant according to the invention.

FIG. 3.—shows an electric diagram of the preferred circuit architecture of FIG. 2, together with the impedance of the electrodes, and the Thévenin equivalent circuit that the implant perceives.

FIG. 4.—shows an electric diagram of another preferred embodiment of an implant circuit according to the invention, for performing measurements with a capacitive transducer.

FIG. 5.—shows an electric diagram of another preferred embodiment of an implant circuit according to the invention, for performing measurements with a resistive transducer instead of a capacitance transducer.

FIG. 6.—shows an electric diagram of another preferred embodiment of an implant circuit according to the invention, for performing voltage measurements.

FIG. 7.—shows an electric diagram of another preferred embodiment of an implant circuit according to the invention, for performing ion concentration measurements based on an ISFET. Ionic conductance is indicated by means of a dotted line.

FIG. 8.—shows an electric diagram of another preferred embodiment of an implant circuit according to the invention, for performing measurements of the phosphorescence of a transducer material. Light transmission is indicated with arrows.

FIG. 9.—shows a schematic representation of a cross-sectional view of another preferred embodiment of the invention, that comprises a capsule material capable of transducing a magnitude of interest (e.g. oxygen concentration) into an optical property (e.g. phosphorescence). Light transmission through the capsule material is represented by means of a broken line.

FIG. 10.—shows an electric diagram of another preferred embodiment of an implant circuit according to the invention, for performing measurements of materials that do not exhibit phosphorescence.

FIG. 11.—shows an electric diagram of the circuit of FIG. 2 with impedance models for the implant and the reading unit electrodes, and an impedance two-port model for the living tissues that couples the reading unit to the implant.

FIG. 12.—shows a graph illustrating the fitting to an exponential decay function by using least squares fitting. Fitting is performed on the average of 100 recordings of the sensed voltage.

FIG. 13.—shows a schematic representation of an implantable sensing system according to the invention, including the electric diagram of the reading unit that allows recording the sensed voltage with the same pair of electrodes used for delivering the interrogation signals.

FIG. 14.—shows an electric diagram of a simplified implant circuit architecture together with the resistances model for the living tissues and the generator of the reading unit.

FIG. 15.—shows examples of unbalanced current amplitude between semi-cycles at different voltage amplitudes applied by the reading unit at: (a) As measured at the reading unit, and (b) as measured at the implant.

FIG. 16.—shows a schematic representation of an embodiment of the invention adapted for measuring lung conductivity for monitoring congestive heart failure.

FIG. 17.—shows a schematic representation of an embodiment of the invention intended to measure blood pressure in vessels.

FIG. 18.—shows two electric diagrams of other preferred embodiments of the implant including a current controlled discharge network, wherein in Figure A the discharge network includes a current controlling device, such as a current-limiting diode (CLD); and in Figure B the discharge network includes a JFET current limiter.

FIG. 19.—shows several flow charts illustrating proposed modes of operation for interrogating the implants performed at the remote unit. In particular Figure A illustrates the mode of operation 1A, Figure B illustrates the mode of operation 1B and Figure C illustrates the mode of operation 2.

### PREFERRED EMBODIMENTS OF THE INVENTION

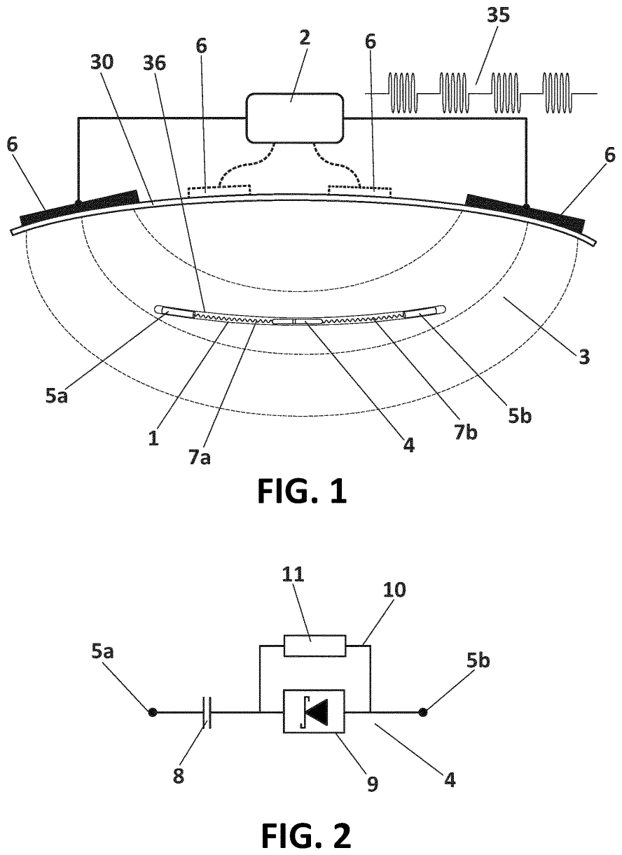

FIG. 1 shows schematically an implantable sensing system according to the invention, comprising an electronic implant (1) implanted in a medium (3) for example a tissue of a living body, and a reading unit (2) adapted to obtain measurements from the implant (1).

The electronic implant (1) comprises an implant circuit (4), at least two electrodes (5a,5b) and two coiled metallic wires (7a,7b) connecting the electrodes (5a,5b) with the implant circuit (4). The implant (1) is configured as an elongated body wherein the implant circuit (4), the two coiled metallic wires (7a,7b) and the electrodes (5a,5b) are linearly arranged, and the electrodes are placed at opposite ends of the elongated body, as shown in FIG. 1.

The implant (1) might be constructed as a flexible tubular body (36) made of biocompatible silicone material, and a hermetic capsule housing the implant circuit, wherein the capsule, the electrodes and the coiled metallic wire, reside within the tubular body.

In the embodiment of FIG. 1, the reading unit (2) is a battery powered hand-held external unit incorporating electrodes (6) that are manually placed on the skin (30) of a subject over the location of a selected implant (1) to be read. The reading unit (2) generates an interrogation signal comprising at least one burst (35) of alternating current, suitable to reach the implant (1) by volume conduction (represented in FIG. 1 by three dashed lines) through the medium (3).

In other embodiments, a plurality of electrodes (6) of the reading unit (2) are placed on a body region where multiple implants (1) are deployed, and a selective interrogation process is automatically performed by means of electronic switching mechanisms, such as relays or analog multiplexors, such that several implants are sequentially interrogated.

In FIG. 1, the reading unit (2) is represented as an external system, however, in other embodiments the reading unit (2), or part of it, is implantable. For example, the reading unit might consist of a subcutaneously implanted sub-unit, adapted to generate bursts (35) of current and for taking measurements, and an external sub-unit adapted for processing data wirelessly transmitted by the implanted sub-unit. The external sub-unit might be adapted for representing measurements and for generating alarms. A conventional smartphone, tablet or similar programmable device, can be adapted for that use.

FIG. 2 shows a basic architecture of the implant circuit (4), that comprises a capacitor (8) and a device of asymmetric conductance (9) capable of rectifying an alternating current (for example a Schottky diode, a LED or a p-n junction of a transistor), such as the capacitor (8) and the device of asymmetric conductance (9) are both connected in series between two electrodes (5a,5b) as shown in FIG. 2. The implant circuit (4) further comprises a discharge network (10) connected in parallel with the device of asymmetric conductance (9).

The discharge network (10) includes at least one electrical or electronic component, a resistor (11) in the example of FIG. 2, which allows the capacitor discharge bypassing the device of asymmetric conductance (9). As a burst (35) of alternating current reaches the implant (1) by volume conduction, the current is rectified by the device of asymmetric conductance (9) and charges the capacitor (8). When the burst of current ends, the capacitor (8) discharges through the discharge network (10) and through the body medium (3). The capacitor (8) prevents that dc currents flow through the implant (1), as these dc currents would cause irreversible electrochemical reactions at the implant electrodes (5a,5b), that in turn would damage both the electrodes and the living tissues.

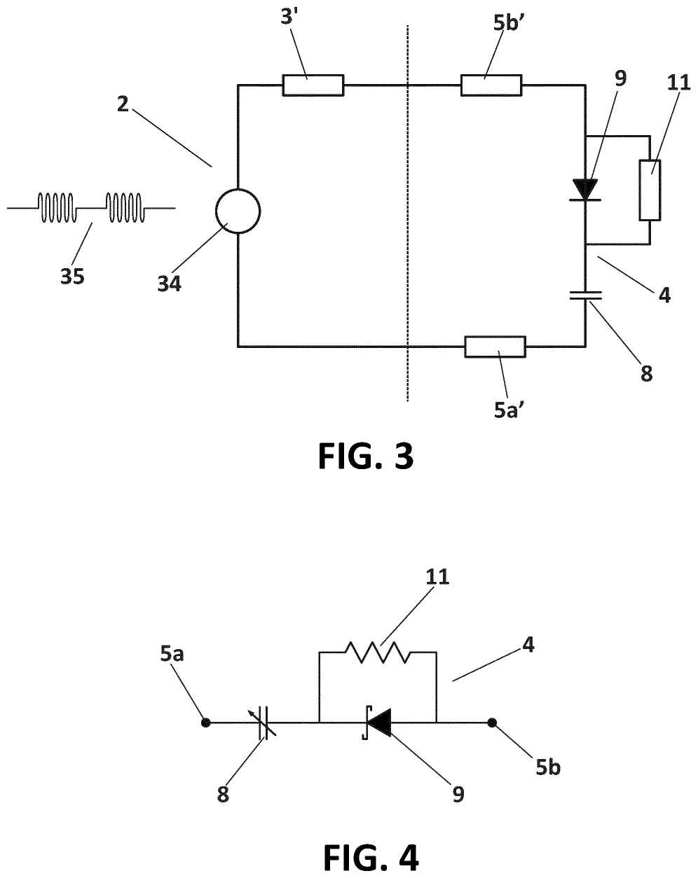

FIG. 3 illustrates the Thévenin equivalent circuit of the medium (3) that surrounds the implant and the electric field. The circuit of FIG. 3 shows the impedance that the implant circuit (4) perceives, including the impedance (5a′,5b′) of the electrodes (5a,5b), and the equivalent impedance (3′) of the medium (3), together with the equivalent voltage source (34) that models the presence of the electric field generated by the current bursts (35). The impedance of the equivalent circuit (Z_{T}) corresponds to the impedance across the implant electrodes, that is, the impedance (3′) of the medium. This impedance is determined by the passive electrical properties of the medium (i.e. the living tissues) and by the geometry of the implant and its electrodes (5a,5b). In some cases it is acceptable to model it as a resistance.

The behavior of the circuit is determined by the impedance of the medium, both during the delivery of the burst (capacitor charging) and afterwards (capacitor discharging). If the components of the implant circuit (4) have known values, it is possible to measure the impedance of the medium by characterizing the behavior of the circuit using the interrogation methods described below.

The voltage source (34) models the presence of the electric field caused by the delivery of the high frequency current bursts (35). Since the involved media are linear and passive, the waveform of this voltage source will be equivalent to that of the applied current or voltage. The amplitude of this source will be a small fraction of the amplitude of the voltage at the current injecting electrodes of the reading unit (2). Coarsely, the maximum amplitude of the equivalent voltage source (34) will be the amplitude of the electric field at the implant location times the distance between the centers of the implant electrodes. This amplitude will be scaled by the cosine of the angle between the electric field and the direction defined by the implant electrodes.

The impedance (5a′,5b′) of the electrodes (5a,5b), can be modeled as a non-linear resistance in parallel with a capacitance. The non-linear resistance accounts for current conduction across the electrode by means of electrochemical reactions. For metal electrodes this resistance can be considered to be very large, in the order of hundreds of kiloohms or megaohms, when the voltage across the electrode is below a threshold in the order of some hundreds of millivolts. The capacitance accounts for the capacitance of the so-called double-layer that forms at the interface between the electrode and the medium. This capacitance is in the order of 10 ρF/cm² in metal electrodes with a smooth surface.

Two preferred interrogation methods are envisioned for the implants:

1.—Operation Based on the Charging or Discharging of the Capacitor.

During the delivery of a burst (35) of high frequency electric current, the capacitor (8) is charged by the rectified current generated by the device of asymmetric conductance (9) that produces a change in the passive behavior of the circuit that can be detected by the reading unit (2) by measuring the voltage or current signals. Once the burst (35) ends, the discharging capacitor (8) causes a voltage across the implant electrodes (5a,5b) that can also be detected by the reading unit (2) by measuring the voltage signal picked up with its electrodes (6). In both phases (i.e. during the burst and after the burst), the time course of the signals depends not only on the characteristic values of the device of asymmetric conductance (9) and the capacitor (8) but also on the characteristic values of the mechanism to discharge the capacitor (8) and on the impedance across the circuit terminals, which consists in the series combination of the impedance of the electrodes (5a′,5b′) and the impedance (3′) of the living tissues. The reading unit (2) can produce measurements of the magnitude of interest by processing either the signals during the burst (35) or the signals after the burst (7). The reading unit (2) can also combine results from both stages in order to improve the accuracy of the measurement.

2.—Operation Based on the Non-Linear Behavior of the Circuit.

During the application of the burst (35) of high frequency electric current, the non-linear behavior of the device of asymmetric conductance (9) induces a current unbalance between positive and negative semicycles of the injected signal. This current unbalance depends not only on the electrical characteristics of the implant components and the living tissue but also on the applied voltage magnitude. The reading unit (2), by performing and processing voltage and current measurements using different excitatory magnitudes, can characterize the non-linear behavior of the device of asymmetric conductance (9) and produce measurements of the magnitudes of interest.

In a preferred embodiment the system is used to measure the electrical impedance or conductance of the tissues surrounding the implant. The implant circuit used for measuring the impedance of surrounding tissues can simply consist of a capacitor in series with the parallel combination of a diode and a resistor.

From the measured impedance it is possible to extract the impedivity of the medium by scaling the impedance according to the cell constant (k). The cell constant is a geometrical factor, typically expressed in units of (m⁻¹) but also sometimes expressed in units of (m) that can be obtained by numerical methods or that can be obtained by measuring a medium of known admittivity (i.e. by calibration). If the measured impedance can be approximated as a resistance (Rₘ), then it will be possible to use the cell constant to extract the conductivity (σ=k/Rₘ) or the resistivity of the medium (ρ=Rₘ/k).

If the interrogation method is based on discharging the implant capacitor (see below), and the impedance across the implant electrodes can be modeled as a resistance (R_{T}), then the time constant (τ_{D}) will correspond to τ_{D}=C(R_{T}+R_{D}) where C is the capacitance of the capacitor of the implant and R_{D} is the resistance of the discharging element of the implant. Hence the resistance measurement (Rₘ) will be simply calculated as: Rₘ=(τ_{D}/C)−R_{D}.

Different magnitudes can be transduced into capacitance. For instance, a large group of pressure sensors is based on isolated conductors that deform under pressure thus becoming closer and hence increasing their mutual capacitance

FIG. 4 shows a preferred embodiment with the same circuit architecture of FIG. 2, but adapted for performing measurements based on sensors whose capacitance depends on the magnitude of interest. In this embodiment, the electrical or electronic component of the discharge network (10) is also a resistor (11) of fixed value, and the capacitor (8) is a transducer whose capacitance is variable depending on a physical and/or chemical condition of the medium (3). Preferably, the capacitor (8) is a pressure capacitor, whose capacitance depends on the pressure applied to a part of the capacitor. The device of asymmetric conductance (9) is a Schottky diode, that is preferred due to its low forward voltage drop that allows shorter implants or lower electric field amplitudes.

Since the transducer capacitance is in series with the uncontrollable capacitance of the electrodes (see FIG. 3), it is required that the capacitance value of the transducer (8) is significantly smaller than that of the electrodes (5a,5b), so that the capacitance value of the series combination is dominated by that of the transducer.

The capacitance of metallic electrodes with a smooth surface is in the order of 10 ρF/cm². Therefore, assuming that the implant electrodes (5a,5b) have a surface area in the order of 1 mm², the above requirement would imply that capacitance value of the transducer has to be much lower than 100 nF.

On the other hand, the capacitance of the transducer needs to be much larger than the possible parasitic capacitances in the implant. These parasitic capacitances can be expected to have values in the order of picofarads.

Therefore, from the above it can be concluded that, in a preferred embodiment of the implant (1) consisting of a thin and flexible elongated body, with a length ranging from a few millimeters to a very few centimeters, and with two electrodes (5a,5b) at opposite ends, the optimal values for the capacitance of the transducer will be within the range: 10 pF to 10 nF.

Similarly, when the interrogation method is based on the discharge of the capacitance of the transducer, it will be advantageous to select a discharge network (10) with a resistance value much higher than the impedance magnitude of the tissues during the discharge. For soft living tissues and the dimensions of the preferred embodiment of the preceding paragraph, those impedance magnitudes will range from about 100Ω to 10 kΩ. And, therefore, it can be anticipated that it will be optimal to select resistors with a resistance between 1 kΩ to 10 MΩ. Larger resistances probably will not be optimal because: 1—they will be comparable to parasitic resistances in the implant and 2—they will cause long charging and discharging times that will lengthen the measurement time.

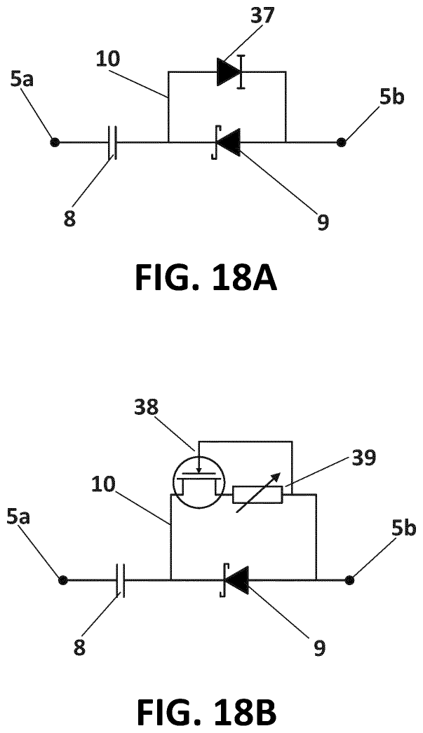

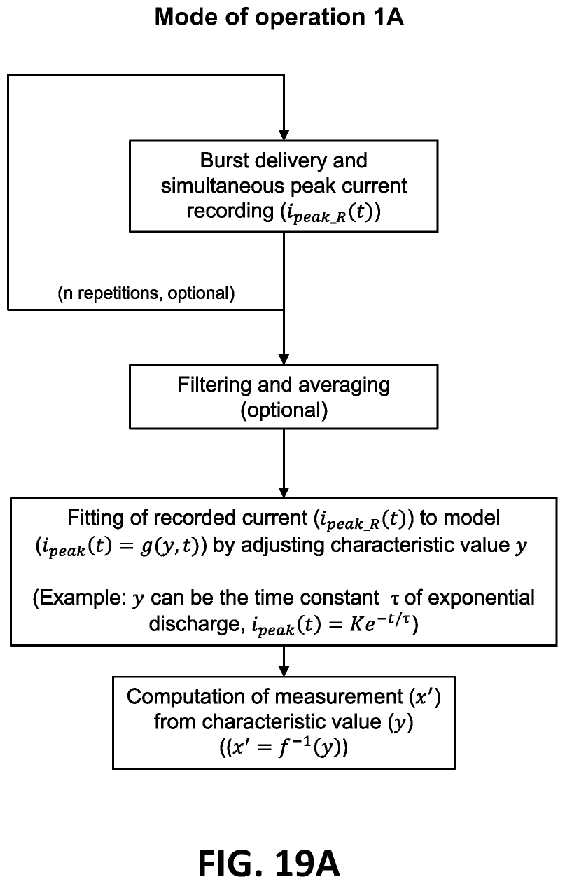

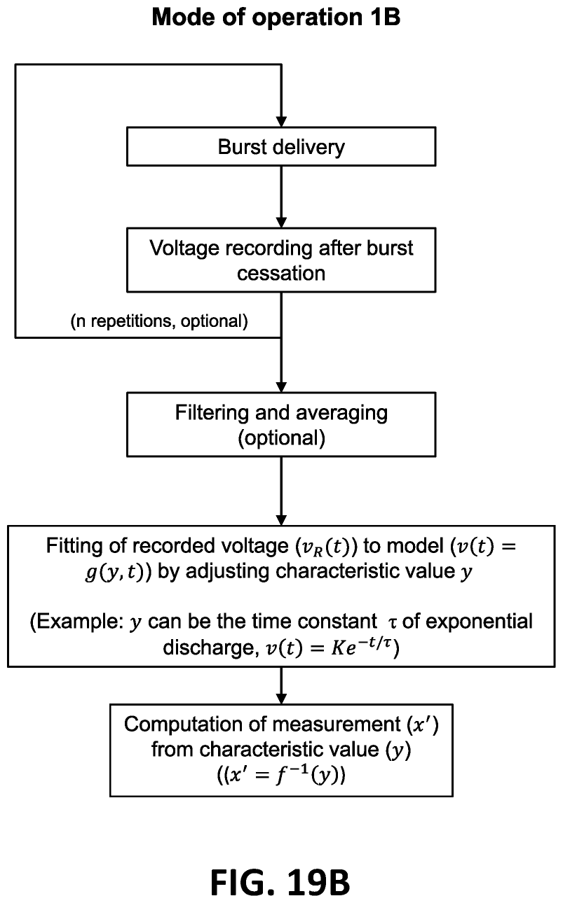

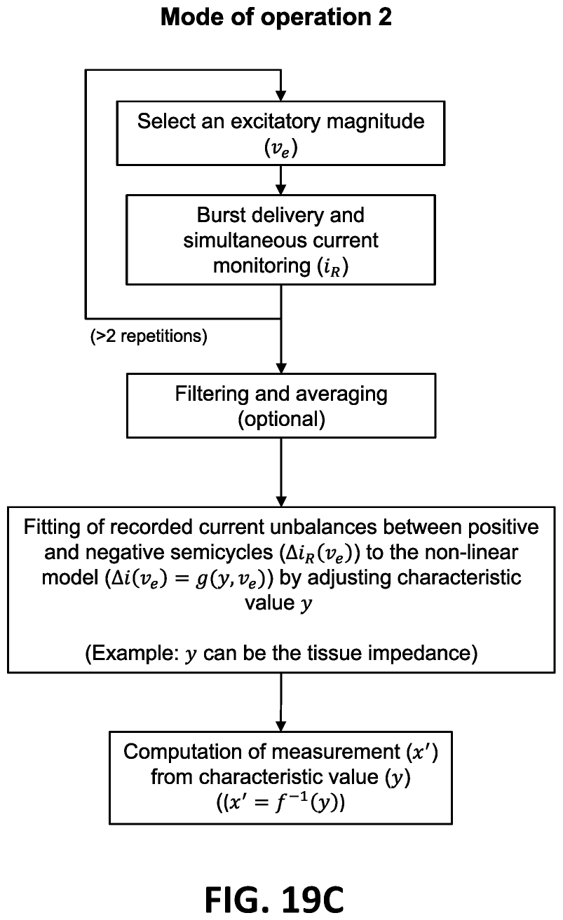

Alternatively, instead of using a discharge network with a controlled resistance, it can be implemented a discharge network of controlled current. In this preferred embodiment, the electric or electronic component of the discharge network (10) is a current controlling device, such as a current-limiting diode (CLD) (37) (represented in FIG. 18A) or a JFET current limiter (38) that controls the discharge current of the capacitor and renders it independent of the impedance of the medium. That current controlling device may be of nominal current like the CLD (37), or may be configured to depend on a measurand. For instance, in the latter case (represented in FIG. 18B), the resistor (39) in a JFET current limiter (38) may be a resistive transducer.

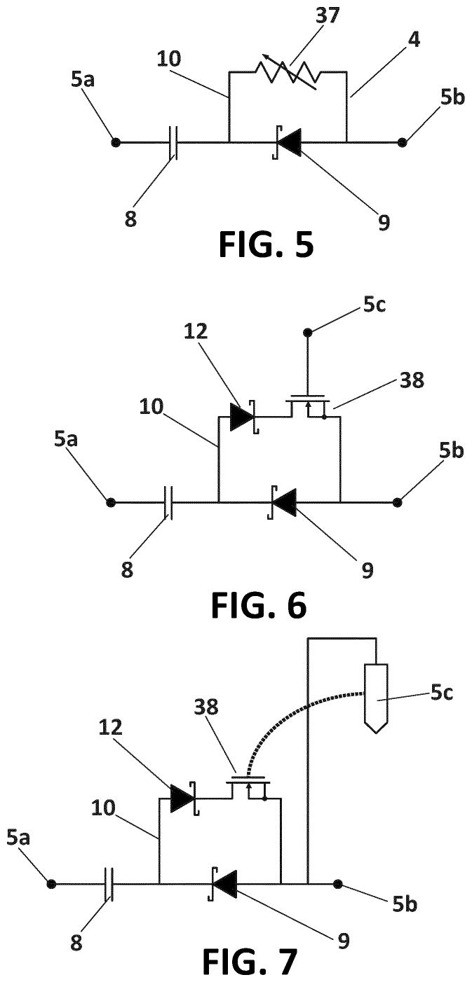

FIG. 5 shows another preferred embodiment with the same circuit architecture of FIG. 2, but adapted for performing measurements based on sensors whose resistance depends on the magnitude of interest. In the embodiment of FIG. 5, the capacitor (8) has a fixed nominal capacitance, the device of asymmetric conductance (9) is a Schottky diode, and the electric or electronic component of the discharge network (10) is a resistive transducer (37), for instance a thermistor or a Light Dependent Resistor (LDRs). Thermistors are known two-terminal semiconductor components that non-linearly transduce temperature into resistance.

FIG. 6 shows an embodiment of the implant for measurement of biopotentials, wherein the capacitor (8) has a fixed nominal capacitance, the device of asymmetric conductance (9) is a Schottky diode and the electronic component is a transducer configured as a transistor (38) whose gate or base terminal is in contact with the medium (3) through a third electrode (5c). The conductance of the transistor (38) depends on the voltage at that third electrode (5c) with respect the other two electrode (5a, 5b). Preferably, the discharge network (10) also includes an auxiliary diode (12) (also a Schottky diode) connected in series with the transistor (38) to prevent conduction when the main diode (9) is forward biased. The anode of the auxiliary diode (12) is connected with the cathode of the diode (9) and one terminal of the capacitor (8), the cathode of the auxiliary diode (12) is connected with the collector or drain of the transistor (38), and the source or emitter of the transistor (38) is connected with the anode of the diode (9) and with anode electrode (5b). The other terminal of the capacitor (8) is connected with the cathode electrode (5a).

Preferably, the transistor (38) is a MOS transistor or a BJT.

FIG. 7 is a variation of the circuit of FIG. 6 for sensing chemical species instead of voltage. In FIG. 7 the transistor (38) is a ChemFet transistor or an Ion Selective Field Effect Transistor (ISFET) for measuring concentrations of chemical species. In this embodiment, the gate of the transistor is directly in contact with the medium and a third electrode (5c) used as reference electrode to stablish ionic conductance (represented as a dotted line in FIG. 7) between the gate and the third electrode (5c). This results in a dependency of the drain-source current on the concentration of the chemical species.

Selectivity to the ion of interest is determined by the material of the gate and of its coatings. Membranes are implemented on the gate to creative selective ISFETs for a wide range of ions (e.g. K⁺, Na⁺ and Cl⁻) and other chemical species (e.g. glucose).

Other preferred embodiments of the invention are based on optoelectronic components (e.g. light dependent resistors (LDRs), photodiodes and phototransistors) whose conductance depends on the received light intensity. Some electronic components, such as light emitting diodes (LEDs), are capable of producing light. All these components are readily integrated in the present invention, to implement implants sensitive to the optical characteristics of the medium (3) or the optical characteristic of a material of the implant that can be in contact with the medium (3).

The material to be optically characterized can be the tissue surrounding the implant (e.g. for pulse oximetry) or a material of the implant. This second option is of particular relevance as there are materials that can transduce a wide range of magnitudes of clinical interest (e.g. chemical concentration of specific chemical species) into optical properties (e.g. fluorescence or phosphorescence characteristics).

Therefore, in another preferred embodiment of the invention, the device of asymmetric conductance (9) and/or the electrical or electronic component of the discharge network (10), is a LED, or a light sensitive semiconductor, for example: a photodiode, or a phototransistor whose conductance depends on the received light.

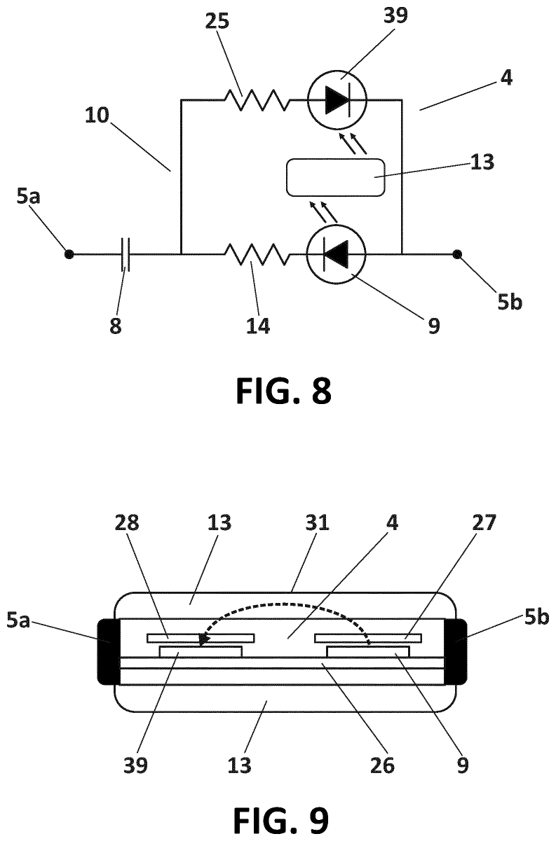

In this range of optical implants, FIG. 8 shows another preferred embodiment of the invention configured for transducing the phosphorescence of an optical reactive material into the conductance of the discharging network. In the embodiment of FIG. 8, the capacitor (8) has a fixed nominal capacitance and the device of asymmetric conductance and the electronic component of the discharge network (10), are constructed in combination as a transducer incorporating an optical reactive material (13), preferably a fluorescence or phosphorescence reactive material, that is arranged in the implant to be in contact with the medium (3).

More in detail, the device of asymmetric conductance is a light emitting semiconductor device (9), for example a Light Emitting Diode (LED) connected in series with a resistance (14). The electronic component is a light sensitive (receiving) conductive device connected in series with a second resistance (25). Preferably in this embodiment, the light sensitive conductive device is a photodiode (39), but it could also consist of an LDR, a phototransistor or a similar component. The second resistance (25) and the photodiode (39) are connected in parallel with the first resistance (14) and the LED (9), such that the cathode of the photodiode (39) is connected with the anode of the LED (9), and one terminal of the resistances (14,25) are connected with one terminal of the capacitor (8), as shown in FIG. 8.

The optical reactive material (13) is arranged in the implant, to receive light from the LED (9) during the capacitor charge, and to irradiate light to the photodiode (39), such as when a current burst ends, the capacitor (8) discharges through the photodiode (39) while this receives light from the optical reactive material (13) due to its fluorescence or phosphorescence property. The optical reactive material (13) is selected, such that its fluorescence or phosphorescence property, is variable depending on a physical or chemical condition of the medium.

FIG. 9 shows a preferred example of constructing the electronic implant of FIG. 8. In FIG. 9 the implant comprises a capsule (31) formed at least in part with the optical reactive material (13). The electronic implant circuit (4) is housed within the capsule (31), and comprises a mounting substrate (26) such as a Printed Circuit Board, and the light emitting semiconductor device (9), the light sensitive conductive device (39), and the resistances (14, 25), are mounted on the substrate (26). The optical reactive material (13) is any known material that can transduce a magnitude of interest (e.g. oxygen concentration) into an optical property (e.g. phosphorescence). Such material could, for instance, consist of a biocompatible hydrogel with phosphorescent dyes with oxygen sensitivity (e.g. Zn²⁺ porphyrins).

The circuit depicted in FIG. 8 is adequate for optical reactive materials exhibiting phosphorescence but, depending on the interrogation method, it may not be adequate for materials not exhibiting phosphorescence because the LED only emits light when the capacitor is being charged, and not when it discharges. A solution to that consists in intercalating a phosphorescent material to illuminate the optical reactive material during the discharge, that is, after the LED ceases emitting light.

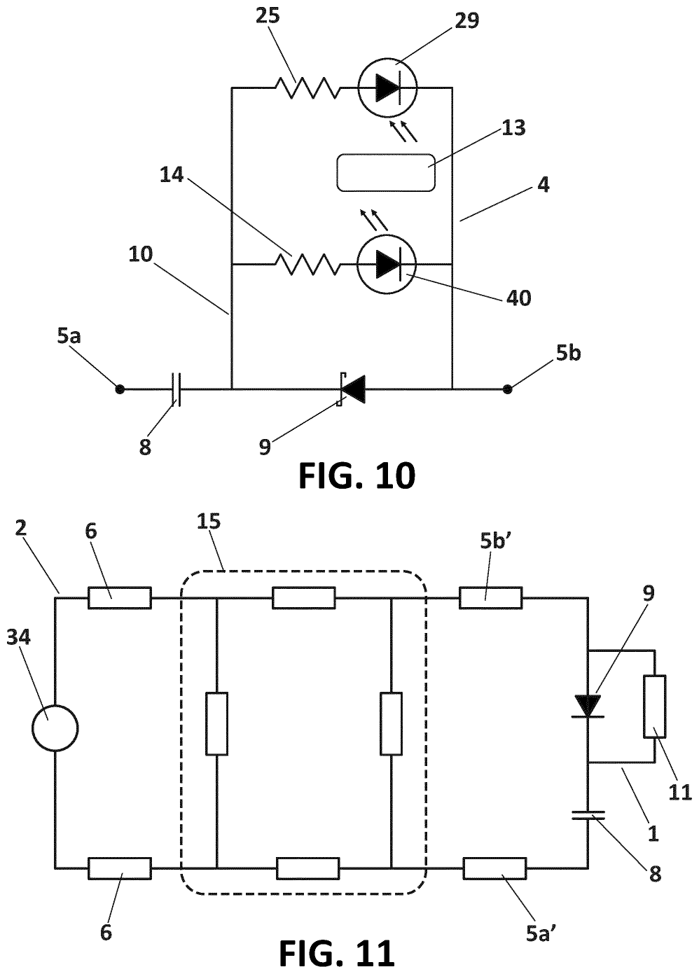

An alternative solution consists in implementing circuits that emit light during the discharge. An example of such circuits is illustrated in FIG. 10, wherein the capacitor (8) has a fixed nominal capacitance, the device of asymmetric conductance (9) is a Schottky diode, and wherein the discharge network (10) comprises a light emitting semiconductor device preferably a LED (40) connected in series with a first resistance (14), and a light sensitive conductive device is for example a photodiode (29) connected in series with a second resistance (25). The second resistance (25) and the photodiode (29) are connected in parallel with the first resistance (14) and the LED (40) and in parallel with the Schottky diode (9), such that the cathode of the photodiode (29) is connected with the cathode of the LED (40), and one terminal of the resistances (14,25) are connected with one terminal of the capacitor (8) and with the cathode of the Schottky diode (9), as shown in the figure. The optical material (13) is arranged to transmit light from the LED (40) to the photodiode (29). The capacitor (8) discharges through the LED (40) so as to emit light during the capacitor discharge. The optical material (13) is a transmitting, reflecting, or refractive optical reactive material. During the discharge, the LED (40) illuminates the transducer material (13) which in turns illuminates—by reflection, transmission, refraction or fluorescence—the photodiode (29) thus determining the discharging rate.

The implant of FIG. 10 can be constructed as the embodiment of FIG. 8.

In order to increase the selectivity of the above implants based on optoelectronic elements, and in particular when it is characterized the fluorescence of the transducer material, it may be advantageous to intercalate optical filters or diffraction grids (27,28) placed on the photoemitter and on the photodetector (photodiode) or as shown in FIG. 9 and FIG. 10, so as to select specific light wavelengths or bands of operation.

Since the optical characteristics of a piece of material not only depend on its intrinsic optical properties but also on its geometry, the above circuits can also be employed to sense changes of geometry. For instance, the transducer material in FIG. 9 can consist in a soft material that compresses under mechanical pressure thus modifying its optical transmissivity between the light emitter and the detector, and hence acting as a pressure transducer.

FIG. 11 facilitates comprehension of the different proposed modes of operation, and illustrates a possible implant architecture according to the invention together with the impedance models for the implant (1) and the reading unit electrodes (6), and an impedance two-port model (15) for the living tissues that couples the reading unit (2) to the implant (1).

Two fundamental modes of operation are envisioned for interrogating the implants:

1. Operation Based on the Charging or Discharging of the Capacitor.

1.A. Operation Based on Monitoring the Load Seen by the Reading Unit During the Delivery of the Burst:

As the capacitor (8) of the implant (1) charges during the delivery of the burst, the ratio between the output voltage amplitude and the output current amplitude of the reading unit (2) increases because, during the semicycles in which the diode (9) is forward biased, the network branch corresponding to the implant progressively draws less current. If the waveform generated by the reading unit (2) is voltage controlled, this is perceived by the voltage generator (34) as a subtle decrease in the amplitude of the current (iₚₑₐₖ(t)) for the semicycles in which the diode (9) is forward biased. This signal, iₚₑₐₖ(t), can be extracted at the reading unit (2) with a peak detector. While the absolute magnitude of iₚₑₐₖ(t) depends on the coupling factor between the reading unit electrodes (6) and the implant electrodes (5a,5b), the time course of relative changes in iₚₑₐₖ(t) does not.

Since the coupling factor is an uncontrollable parameter as it depends on the distance between the reading unit and the implant, it is preferred to base the interrogation on analyzing the time course of relative changes in iₚₑₐₖ(t). The time course of relative changes in iₚₑₐₖ(t) depends on the impedance of the tissues as seen by the implant (Z_{T}), the impedance of the implant electrodes (Z_{E}), the capacitance (Cᵢ) of the implant capacitor, the resistance (R_{D}) of the discharging element of the implant and the characteristics of the diode.

The time course of relative changes in iₚₑₐₖ(t) can be modelled analytically or numerically (e.g. by SPICE simulations) to characterize its dependence on the referred elements. Then, contrasting such characterization with the recorded time course of relative changes in iₚₑₐₖ(t), the reading unit can compute the characterization of an unknown element if the other ones are known. In the specific case in which the equivalent impedance of the tissues can be approximated to behave as a resistance (i.e. Z_{T}=R_{T}) and the impedance of the implant electrodes can be neglected (Z_{E}=0), iₚₑₐₖ(t) will follow an exponential time decay whose time constant (T) will be computable by the reading unit as described in the operation mode 1.B. Then, if the capacitance (Cᵢ) of the capacitor and the resistance (R_{D}) of the discharging element are fixed and known, the reading unit will be able to compute the value of the resistance of the medium.

If the waveform generated by the reading unit (2) is current controlled instead of voltage controlled, analogous computing procedures to those described above can be carried out by the reading unit (2) by processing the subtle increases in the voltage amplitude at the semicycles in which the diode is forward biased (νₚₑₐₖ(t)).

Since the subtle changes in either current or voltage are overlapped on a large signal, the implementation of the method requires detection and processing with a very large dynamic range.

Another inherent limitation of this mode of operation is that it is only useful to measure the resistance or the impedance of the tissues. That is, it is not useful to independently measure neither the capacitance of the implant capacitor nor the resistance of the discharge network.

1.B. Operation Based on Monitoring the Voltage Produced During the Discharge of the Capacitor:

The capacitor (8) of the implant (1), which is charged during the delivery of the burst, discharges after burst cessation through the discharge network (10), the implant electrodes (5a,5b) and the tissues surrounding the implant. This produces a decaying voltage across the implant electrodes that can be sensed distantly by pairs of electrodes in contact with the tissues. In a preferred embodiment, this voltage is sensed across the electrodes (6) of the reading unit (2). By characterizing the sensed waveform, it is possible to compute the capacitance of the implant capacitor (8), the resistance (11) of the discharging element of the implant circuit or the impedance of the tissues. In particular, if the impedance of the tissues can be modeled as a resistance (i.e. Z_{T}=R_{T}) and the impedance of the implant electrodes is neglected, the sensed voltage will be:

ν_{sensed}(t)=αV₀e^{-t/τ}; τ=Cᵢ(R_{D}+R_{T})

where α is the coupling factor between the implant electrodes and the sensing electrodes, V₀ is the initial voltage across the implant electrodes right after burst cessation, Cᵢ is the capacitance of the implant capacitor and R_{D} is the resistance of the discharging element of the implant.

From the above it is obvious that any of the values Cᵢ, R_{T} and R_{D} can be obtained if the value of the time constant (τ) is computed and the other values are known.

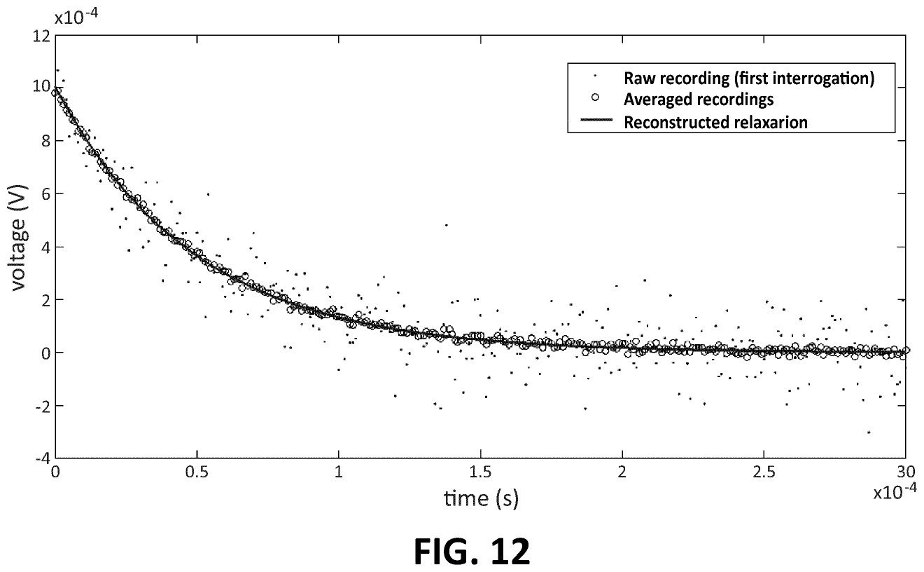

From the sensed voltage (ν_{sensed}(t)) it is possible to extract the value of the time constant (τ) by different known methods. In a preferred embodiment of the invention, the time constant is obtained by fitting the average of multiple recordings of the sensed voltage to a generic exponential decay function (y(t)=Ae^{-γt}) by using least squares fitting. An example of the result of this operation is represented in the graph of FIG. 12. This procedure to obtain an estimation of the time constant provides good results under the presence of noise and interferences.

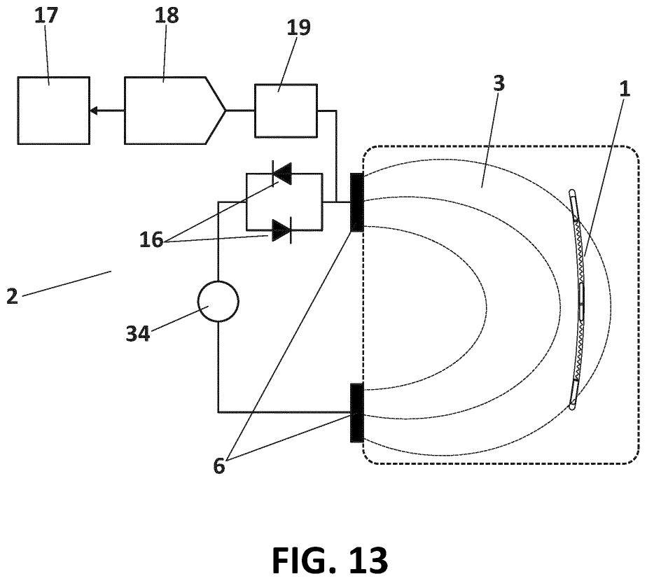

FIG. 13 shows a preferred embodiment of the system of the invention incorporating an implant (1) deployed in a medium (3), and a reading unit (2) to interrogate the implant (1). The reading unit (2) is represented by its electric diagram that allows recording the sensed voltage (ν_{sensed}(t)) with the same electrode (6) pair used for delivering the bursts. The burst is generated by a voltage generator (34) of relatively high amplitude, for example above 5V. After the interrogation signal terminates, the voltage generator (34) disconnects from the electrodes (6) so that it does not short-circuit them.

In the embodiment of FIG. 13, such disconnection is performed by two diodes (16) in antiparallel connection: the diodes (16) allow passage of high magnitude signals during the delivery of the interrogation signal, but behave as an open circuit for the much smaller sensed voltage, which will have a voltage amplitude below 100 mV. A low-pass filter (19) is used to suppress noise and interferences and to prevent saturation of posterior stages. The sensed voltage (ν_{sensed}(t)) is captured with an analog-to-digital converter (18) and later processed by a control and processing module (17) to obtain the time constant as described above.

2. Operation Based on the Non-Linear Behavior of the Circuit

During the application of the burst of high frequency electric current, the non-linear behavior of the diode (9) induces a current unbalance between positive and negative semicycles of the injected signal. This current unbalance depends not only on the electrical characteristics of the implant elements and the living tissues but also on the applied voltage magnitude. The reading unit (2), by performing and processing voltage and current measurements using different excitatory magnitudes, can obtain a set of independent equations from which it can compute the characteristic values of the elements of the system and hence produce measurements of the magnitudes of interest. Here it is illustrated the procedure by detailing a simplified example.

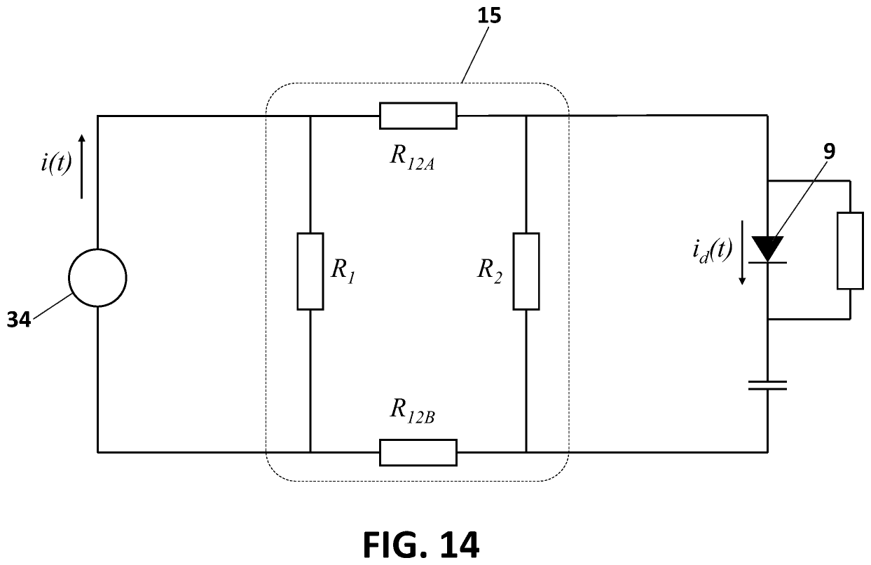

FIG. 14 shows the electrical representation of the system used to illustrate the procedure where the living tissues model (15). At high frequencies, living tissues can be approximated to behave as resistances and the electrode interface impedances can be neglected.

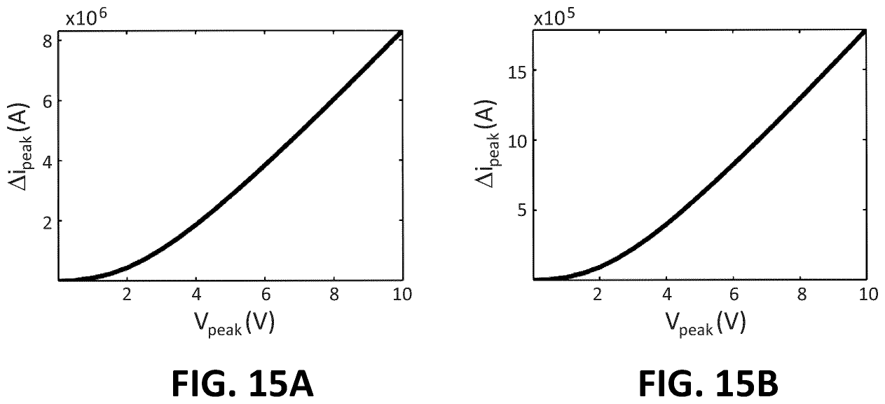

For the illustration scenario (FIG. 14), in the case of a voltage controlled reading unit (2) that delivers sinusoidal bursts, the output current amplitude (iₚₑₐₖ(t)) during the semicycles in which the diode is forward biased (i_{peak|+}) shows larger values than during the complementary semicycles (i_{peak|-}). This results in a current difference between both semicycles (Δiₚₑₐₖ(t)) Because of the non-linear behavior of the diode (9), the current unbalance between semicycles at the reading unit (2) depends on the voltage amplitude (νₚₑₐₖ(t)) (FIG. 15a). The same non-linear behavior is present at the implant side (FIG. 15b).

Since the non-linear behavior of the diode (9) can be well characterized, by measuring the current unbalance between semicycles at the reading unit (2) for different applied voltages, it is possible to detect the non-linear behavior which indirectly determines the current circulating through the implant.

The reading unit (2) is able to compute the value of the resistance values of the living tissue port model (15) based on the current peak unbalance (Δiₚₑₐₖ) and the characterized non-linear behavior of the implant. This can be readily illustrated with the proposed example. Considering very short bursts, the implant can be simplified as a single diode (9) with an idealized behavior: it causes a constant forward voltage drop (ν_{f}) when the diode is forward biased and completely blocks current when it is reverse biased. Then:

$${Δ {i}_{peak} ( {v}_{peak} ) = {i}_{peak} {\vert}_{+} - {i}_{peak} \vert}_{-}$$

$${i}_{peak} {\vert}_{+} = \frac{{v}_{peak}}{R 1} + \frac{{v}_{peak} - {v}_{f}}{{R}_{12}}$$

$${i}_{peak} {\vert}_{-} = \frac{{v}_{peak}}{{R}_{1}} + \frac{{v}_{peak}}{{R}_{12} + {R}_{2}}$$

$$Δ {i}_{peak} ( {v}_{peak} ) = \frac{{v}_{peak -} {v}_{f}}{{R}_{12}} - \frac{{v}_{peak}}{{R}_{12} + {R}_{2}}$$

$$Where {R}_{12} = {R}_{12 A} + {R}_{12 B}$$

Therefore, taking advantage of the non-linearity of the diode, by performing additional measurements at different voltage magnitudes, non-redundant (i.e. independent) equations can be obtained that allow solving the unknowns of the system from which measurements can be produced.

$$Δ i ( {v}_{1} ) = \frac{{v}_{1} - {v}_{f}}{{R}_{12}} - \frac{{v}_{1}}{{R}_{12} + {R}_{2}} \to \frac{1}{{R}_{12} + {R}_{2}} = \frac{\frac{{v}_{1} - {v}_{f}}{{R}_{12}} - Δ i ( {v}_{1} )}{{v}_{1}}$$

$$Δ i ( {v}_{2} ) = \frac{{v}_{2} - {v}_{f}}{{R}_{12}} - \frac{{v}_{2}}{{R}_{12} + {R}_{2}} \to \frac{1}{{R}_{12} + {R}_{2}} = \frac{\frac{{v}_{2} - {v}_{f}}{{R}_{12}} - Δ i ( {v}_{2} )}{{v}_{2}}$$

$${R}_{12} = \frac{{v}_{2} \cdot ( {v}_{1} - {v}_{f} ) - {v}_{1} ( {v}_{2} - {v}_{f} )}{Δ i ( {v}_{1} ) \cdot {v}_{2} - Δ i ( {v}_{2} ) \cdot {v}_{1}}$$

In case of current controlled reading unit, analogous computing procedures to those described above can be carried out by replacing current to voltage terms and vice versa.

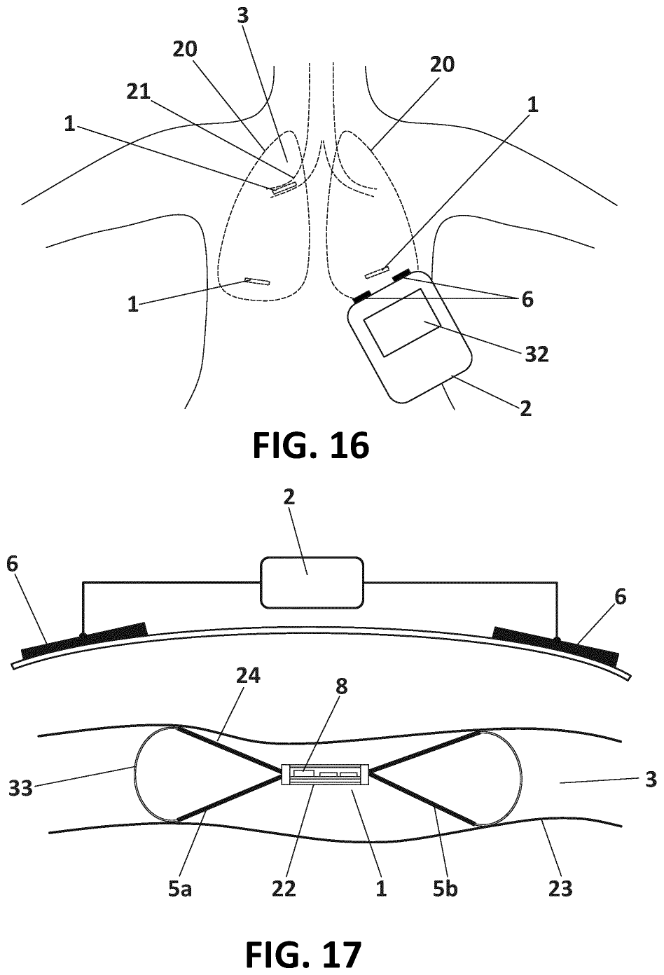

FIG. 16 also illustrates a practical application of the system of the invention, intended to measure lung impedance for early detection of worsening heart failure. In this embodiment, implants (1) according to the invention can be deployed at different possible locations either in the lungs (20) or in nearby tissues to monitor the conductivity of the lungs.

Pulmonary edema, that is, accumulation of fluids in the lungs, occurs during worsening heart failure. Since such accumulation of fluids causes an increase in the conductivity of the lungs, the measurements provided by the implants (1) can be used to determine when heart failure is worsening.

In this embodiment of the invention, the implants (1) are deployed at locations that will result in a higher sensitivity to the conductivity of the lungs than that offered by systems utilizing endovascular electrodes. For instance, the implants (1) can be deployed at the intercostal spaces. There, implants can be easily and safely deployed within the intercostal muscles using percutaneous procedures (i.e. injection). Alternatively, for maximizing sensitivity, the implants can be deployed in the lungs. In the lungs, the implants can be deployed within the parenchyma using a percutaneous procedure or within the airways (i.e. bronchi and bronchioles (21)) through bronchoscopy. In the latter case, for facilitating fixation, the implants (1) are shaped as pulmonary stents.

A reading unit (2) for interrogating the implant here is a battery-powered hand-held unit integrating skin electrodes (6) and a display (32), that the patient positions on his or her skin close to the implant to be interrogated. It can also consist in battery-powered unit shaped as a pod or capsule that is fixed on the skin over the location of the implant, for instance, by a fastener on sticking plaster, and that stores measurements in memory or transmits those measurements by radio to a nearby computerized device such as a smartphone.

FIG. 17 illustrates an alternative embodiment of the implant (1) suitable to be deployed inside an artery or a vein (23) for measuring blood pressure. The implant (1) comprises a capsule (22) having at least a part made of a flexible material that allows pressure transmission from the exterior (caused by blood pressure) to the capsule interior. For example, the flexibility and hermeticity of the capsule (22) can be obtained with a tubular capsule of thin metallic walls of thickness <0.5 mm.

The implant circuit (4) is hermetically housed within the capsule (22), and it can consist in any of the configurations described above suitable for performing measurements with a pressure sensor.

The implant further comprises two electrodes (5a,5b) passing through the capsule (22) and connected to the implant circuit (4), wherein each electrode (5a,5b) is a flexible structure configured to anchor the implant to an artery or vein.

The connection between the electrodes (5a,5b) and the circuit (4) can be performed through hermetic feedthroughs to ensure the hermeticity of the capsule (22). Each electrode is a flexible wire conformed as a loop. An inner part of the electrodes (5a,5b) is covered by an insulating material (24), and an outer part (33) of the electrodes (5a,5b) is exposed to the medium, flowing blood in this case. This configuration for the implant electrodes (5a,5b) facilitates implant deployment, anchorage and extraction. For deployment, the loops can be readily folded within a catheter for enabling minimally invasive implantation through catherization. After deployment, the loops will unfold due to their flexibility or because of shape memory if the wire is made of a metal exhibiting such property such as nitinol and, by pressing the vessel walls, will anchor the implant within the vessel. If required, for minimally invasive implant extraction, the implant can be extracted with a catheter by pulling out with a hook one of the two loops of the implant.

The pressure sensor can be a capacitive pressure sensor. In this case the circuitry of the implant can consist in the circuit illustrated in FIG. 4 for performing measurements with capacitance-based sensors.

The capacitive pressure sensor does not necessarily have to consist in a stand-alone sensor. It can consist in a capacitive pressure sensor formed by using the capsule wall as one of the electrodes of a capacitor. For instance, a dielectric (e.g. air) can be sandwiched within the coaxial structure formed by the semi-flexible capsule wall and an inner, and more rigid, metallic tube or cylinder to form a capacitor whose capacitance increases when the distance between the two metallic parts decreases as the result of pressure.

1. A sensing system comprising at least one implant,
wherein the implant comprises an electronic circuit and at least two electrodes connected to the electronic circuit,
wherein the electronic circuit comprises a capacitor and a device of asymmetric conductance both connected in series between the at least two electrodes, the electronic circuit further comprising a discharge network connected in parallel with the device of asymmetric conductance for a capacitor discharge, wherein the discharge network comprises at least one electrical or electronic
component, and
a reading unit for reading the implant when the implant is deployed in a medium, wherein the reading unit comprises two or more electrodes, an alternating voltage generator to generate an alternating voltage across the two or more electrodes, and a control and processing module, wherein the reading unit is configured for emitting an interrogation signal comprising at least one burst of an alternating current suitable to reach the implant by volume conduction through the medium, wherein the reading unit is adapted for measuring voltage and/or current signals at the two or more electrodes of the reading unit, and wherein the signals depend on operation of the implant during and/or after delivering the at least one burst of the alternating current.

2. The sensing system according to claim 1, wherein the reading unit is adapted to emit bursts of the alternating current at a frequency between 100 kHz and 100 MHz, with bursts duration between 0.1 μs and 10 ms, and a repetition frequency between 0 Hz and 100 kHz.

3. The sensing system according to claim 1, wherein the implant further comprises an elongated and flexible body made of an electrically isolating material, and wherein the electronic circuit is housed within the body, and wherein the implant further comprises two metallic electrodes at opposite ends of the body which are electrically connected to the electronic circuit, and wherein optionally the length of the implant is within the range 0.5 cm-5 cm.

4. The sensing system according to claim 1, wherein the discharge network of the implant further comprises a current controlling device for controlling a discharge current of the capacitor and makes a discharge process independent of impedance of the medium and of the at least two electrodes of the implant.

5. The sensing system according to claim 1, wherein the capacitor, or the device of asymmetric conductance and/or the electrical or electronic component, is a transducer selected such that an operational parameter of the transducer is variable depending on a physical and/or chemical condition of the medium when the implant is deployed in the medium, and wherein optionally the device of asymmetric conductance is a diode, a p-n junction of a transistor or a smart diode.

6. The sensing system according to claim 5, wherein the electrical or electronic component of the discharge network is a resistor of a given nominal value, and wherein the capacitor is a transducer whose capacitance is variable depending on the physical and/or chemical condition of the medium.

7. The sensing system according to claim 5, wherein the capacitor has a given nominal capacitance, and wherein the electronic component of the discharge network is a resistive transducer.

8. The sensing system according to claim 5, wherein a capacitance of the capacitor is within the range 10 pF to 10 nF, and wherein a resistance of the discharge network is within the range 1 kΩ to 10 MΩ.

9. The sensing system according to claim 5, wherein the sensing system is suitable for measuring biopotentials, wherein the capacitor has a given nominal capacitance, and wherein the discharge network comprises a transistor, whose gate or base terminal is arranged to be in contact with the medium by means of a third electrode, wherein conductance of the transistor depends on a voltage at the third electrode.

10. The sensing system according to claim 5, wherein the sensing system is suitable for measuring chemical species, wherein the capacitor has a given nominal capacitance, and wherein the discharge network comprises a ChemFet transistor or an Ion Selective Field Effect Transistor adapted such that a gate of the transistor is in contact with the medium when the implant is deployed in the medium, and wherein conductance of the transistor depends on a concentration of chemical species at the gate of the transistor.

11. The sensing system according to claim 5, wherein the capacitor has a given nominal capacitance, and the implant further comprises an optical reactive material, preferably a fluorescence or phosphorescence variable material, arranged in the implant to be in contact with the medium when the implant is deployed in the medium, and wherein an optical property of the transducer is variable depending on the physical or chemical condition of the medium, wherein the device of asymmetric conductance is a light emitting semiconductor device, wherein the discharge network comprises a light sensitive conductive device, wherein an optical material is arranged to transmit, reflect or refract light from the light emitting semiconductor device to the light sensitive conductive device.

12. The sensing system according to claim 5, wherein the capacitor has a given nominal capacitance, and the implant further comprises a transmitting, reflecting, or refractive optical reactive material, wherein the optical reactive material is arranged in the implant to be in contact with the medium when the implant is deployed in the medium, and wherein the discharge network is connected in parallel with the device of asymmetric conductance, and comprises a light emitting semiconductor device, wherein the light emitting semiconductor device emits light during the capacitor discharge, and a light sensitive conductive device connected in parallel, and wherein the optical reactive material is arranged to transmit, reflect or refract light from the light emitting semiconductor device to the light sensitive conductive device.

13. The sensing system according to claim 11, further comprising optical filters or diffraction grids placed on the light emitting semiconductor device or on the light sensitive conductive device to select specific light wavelengths or bands of operation.

14. The sensing system according to claim 5, wherein the sensing system is suitable to be implanted inside an artery or a vein for measuring blood pressure, further comprising a capsule having at least a part made of a flexible material that allows pressure transmission from an exterior of the capsule to an interior of the capsule, and wherein the electronic circuit is housed within the capsule, the implant further comprising the at least two electrodes passing through the capsule and connected with the electronic circuit, wherein each electrode of the at least two electrodes is a flexible structure configured to anchor the implant to the artery or vein, and wherein the capacitor is a capacitive pressure sensor, such as the capacitor and the flexible part of the capsule are arranged relative to each other wherein the capacitance of the pressure sensor depends on the blood pressure.

15. The sensing system according to claim 5, wherein the discharge network comprises a current controlling device incorporating a transducer wherein current control depends on a parameter of the transducer, and wherein optionally the current controlling device is a JFET transistor and a resistive transducer wherein a source of the JFET is connected to one terminal of the resistive transducer and a gate of the JFET transistor is connected to the other terminal of the resistive transducer.

16. The sensing system according to claim 1, wherein the reading unit is additionally adapted for processing a sensed voltage and/or current, to calculate a capacitance of the capacitor, a resistance of the discharging network or impedance of the medium surrounding the implant when the implant is deployed in the medium, and for processing a calculated value to obtain a measurement of interest.

17. The sensing system according to claim 16, wherein the reading unit is adapted for reading the implant by monitoring a time course of relative changes of a burst current amplitude iₚₑₐₖ as the capacitor charges, for fitting recorded variations of the time course of relative changes of the burst current amplitude iₚₑₐₖ to a model by adjusting a characteristic value, and for calculating a desired measurement from the characteristic value.

18. The sensing system according to claim 16, wherein the reading unit is adapted for reading the implant by monitoring the voltage across the two or more electrodes during the capacitor discharge after a burst cessation, for fitting a recorded voltage waveform to a model by adjusting a characteristic value, and for calculating a measurement from the characteristic value.

19. The sensing system according to claim 16, wherein the reading unit is adapted for reading the implant by delivering bursts of different amplitude and monitoring the current unbalances between positive and negative semicycles, for fitting recorded unbalances to a model by adjusting a characteristic value, and for calculating a measurement from the characteristic value.

20. The sensing system according to claim 1, wherein the reading unit is an external battery powered hand-held unit, or wherein the reading unit or at least a part of the reading unit comprises an implantable sub-unit adapted for reading the implant, and an external sub-unit adapted for presenting information related to the readings of the implantable sub-unit, and wherein the two sub-units are wirelessly communicated.
